(19) United States
(12) Patent Application Publication
(10) Pub. No.: US 2022/0264887 A1
(43) Pub. Date: Aug. 25, 2022

(54) Foam Formulations For Decontamination Of Surfaces With Minimum Wastewater

(71) Applicant: The Trustees of Columbia University in the City of New York, New York, NY (US)

(72) Inventors: Ponisseril Somasundaran, New York, NY (US); Partha Patra, New York, NY (US)

(21) Appl. No.: 17/560,640

(22) Filed: Dec. 23, 2021

Related U.S. Application Data
(60) Provisional application No. 63/131,975, filed on Dec. 30, 2020.

(51) Int. Cl. A01N 59/00; A01N 25/16; A01N 25/30; A01N 33/12; A01P 1/00; A61L 2/18

(52) U.S. Cl. CPC A01N 59/00; A01N 25/16; A01N 25/30; A01N 33/12; A01P 1/00; A61L 2/18; A61L 2101/18

(57) ABSTRACT

Provided are foams and foamable formulations for the decontamination of surfaces, such as disinfection from COVID-19. The formulations can allow deposition of a uniform layer of foam on a given surface without spontaneous drainage of water and with near zero flowability. The foam can adhere to slanted surfaces and surfaces comprised of a variety of materials, including wood, glass, and stone, enabling safe and effective viral decontamination in a number of settings.

### RELATED APPLICATIONS

The present application claims priority to and the benefit of U.S. patent application No. 63/131,975, “Foam Formulations For Decontamination Of Surfaces With Minimum Wastewater” (filed Dec. 30, 2020), the entirety of which application is incorporated herein by reference for any and all purposes.

### GOVERNMENT RIGHTS

This invention was made with government support under 2026740 awarded by the National Science Foundation. The government has certain rights in the invention.

### TECHNICAL FIELD

The present disclosure relates to the field of disinfecting foam formulations.

### BACKGROUND

Disinfection of surfaces is of importance in a variety of settings, including commercial, residential, and medical settings. Surface disinfection is of particular concern during the COVID-19 pandemic because of possible surface-to-surface transmission of the virus.

At present, a number of disinfectants (including quaternary ammonium compounds, hydrogen peroxide, alcohol-based solutions, and others) are recommended for surface disinfection applications. Some disinfectants, however, can produce gases or other byproducts, corrode surfaces, and/or have highly flammable properties. Further, many existing disinfectant formulations are resource-intensive, as the formulations use significant amounts of disinfectant as well as significant amounts of water and other ingredients.

Accordingly, there is a long-felt need in the art for effective and environmentally friendly decontamination formulations and related methods. This need is highlighted by the need for decontamination during the ongoing coronavirus pandemic as well as by the need for decontamination following hurricanes and floods, both of which give rise to environments that act as breeding grounds for microbes and viruses.

### SUMMARY

The disclosed technology provides, inter alia, a hypochlorite-based foam formulation for the decontamination of surfaces, e.g., decontamination of COVID-19. An example of the disclosed formulations incorporates cocamidopropyl betaine (CAPB) and sodium dodecyl sulfate (SDS) as the main surfactants, along with surfactin for its antiviral properties, to produce a hypochlorite formulation (suitably a foam, but which can also be in the form of a gel or cream) that is safe for the environment and repeated use on various surfaces. Foam texture, described as exhibiting characteristics ranging anywhere from wet to completely dry foam, was adjusted to enable an optimum residence time of water film on a given surface after breakage of the foam, thereby minimizing any fumes generated by the hypochlorite solution. When deposited using a sprayer, the disclosed formulation forms a uniform layer on the receiving surface without spontaneous drainage of water and near zero flowability. The foam can adhere to slanted surfaces and surfaces comprised of a variety of materials, including wood, glass, and stone, enabling safe and effective viral decontamination in various settings.

In meeting the described long-felt needs, the disclosed technology first provides an aqueous foam (which can be characterized as wet to semi-wet), comprising: at least one surfactant; at least one defoamer; optionally at least one viscosity modifier, and optionally, at least one disinfectant, the at least one surfactant, the at least one defoamer, and the at least one optional disinfectant being present such that (a) the foam is characterized as breaking within about 30 minutes of application to a surface under exposure to ambient environmental conditions, (b) the foam exhibits an essentially uniform deposition along a surface to which the foam is applied, (c) the foam forms a fluid layer at an interface between the foam and a surface to which the foam is applied, (d) any combination of (a), (b), and (c).

Also provided are methods comprising: formation of a foam according to the present disclosure, wherein the formation of the foam comprises foaming a foamable base formulation; and effecting deposition of the foam onto a surface, the deposition of the foam optionally effecting disinfection of the surface.

Further disclosed are foamable base formulations, comprising: at least one surfactant; at least one defoamer; and optionally, at least one disinfectant, the at least one optional disinfectant, the at least one surfactant, and the at least one defoamer being present such that the foamable formulation is capable of being foamed so as to give rise to a foam according to the present disclosure.

Additionally provided are systems, comprising: a vessel configured to contain a foamable base formulation according to the present disclosure; and an aeration train, the aeration train configured to effect aeration of the foamable base formulation so as to give rise to an aqueous foam according to the present disclosure.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1. Surface tension values of SDS and CAPB at 1:1 wt. % ratio in the presence and absence of hypochlorite solution; blue circles indicate absence of hypochlorite, orange and black circles indicate data for surfactants presence of hypochlorite at 1 and 10 wt. % CL respectively.

FIG. 2. Surface tension values as a function of time for surfactant formulations prepared from a batch with 1.5 wt. % CAPB, 1.5 wt. % SDS, and 0.7% CL (Hypochorite).

FIG. 3. Surface tension for surfactant formulations—3 wt. % CAPB, SDS, and Surfactin and 0.8% CL (Hypochlorite) CAPB:SDS:Surfactin=1:1:1. Series 1 and 2 indicates two arbitrarily prepared different dilutions—surfactant concentration <CMC—for the surfactant formulations.

FIG. 4. Cumulative foam height as a function of foaming time at airflow rate of 100 mL/min. The data presented, based on duplicate tests, are within ±10% of error.

FIG. 5. Foam breaking time for foams prepared from different formulations. The foam produced with 3 wt. % surfactants and at 100 mL/min was placed on glass slides, and breaking time of foam was considered from the time of deposition until no individual bubbles were observed. Data presented are within of 10-15% error, and *FB: Foam Breaker—dodecanol (0.5% v/v) and Polyethylene glycol (0.15 v/v)

FIG. 6. (Top) Bubble size of foam formed with SDS+CPAB mixture and (Bottom) Bubble size of the foam formed with a SDS, CAPB and surfactin mixture.

FIGS. 7A-7B—FIG. 7A Top Left—foam sprayer used to generate foam, Top Middle—foam deposition on a vertical glass surface (window), and Top right—image take for the foam deposited onto the window using a camera. FIG. 7B Bottom left—Foam deposited onto a glass slide, and Bottom right—Phase contrast microscopy image of the foam deposited onto a glass slide.

FIG. 8. Raman spectra of 1% solutions of sodium hypochlorite (10-15% NaClO, reagent grade, Aldrich) and sodium perchlorate (NaClO₄, ACS certified, Fisher)

FIGS. 9A-9B—FIG. 9A Raman spectra of 30-min-aged foams and dry residues of (1.5 wt % SDS+1.5% wt CAPB+1% NaClO) and, for comparison, of (1.5 wt % SDS+1.5% wt CAPB). FIG. 9B Optical image of a foam showing a typical focusing spot for Raman measurements.

FIG. 10. Raman spectrum of formulation (1.5 wt % SDS+1.5 wt % LAPB+1 wt % NaClO) measured 24 h after the preparation. The circle shows traces of the ClO— peak.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints, 2 grams and 10 grams, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

The disclosed technology provides, inter alia, hypochlorite-based foam formulations for the decontamination of surfaces. The formulations can incorporate green surfactants—including surfactants that demonstrate antiviral properties—and enable a shorter residence time of water film on a given surface. The foam can adhere to slanted surfaces and surfaces comprised of a variety of materials, including wood, glass, and stone, enabling safe and effective viral decontamination in various settings.

Deploying hypochlorite-based foam on a surface provides distinct advantages, as the amount of hypochlorite being used is reduced, resulting in a reduced volume of hypochlorite fumes being inhaled and/or seeping into the ground.

Desirable Criteria

Certain desirable (but non-limiting) criteria for such a foam include:

1) Foam deposited onto a surface must form a thin (hundreds of nanometers to several millimeters) layer of fluid phase—containing hypochlorite—at the foam-surface interface, and for a duration desired to deactivate the virus.

2) Uniform deposition of foam across a surface—no patchy regions,

3) The cross section of the deposited foam from the top to the surface is formed of a few layers of bubbles placed on top of each other, as opposed to the formation of multilayer “lumpy” foam.

4) Foam adheres to slanted or roof surfaces instead of dripping off, and

5) Foam collapses/breaks after a desired period—until the virus is deactivated.

Objectives

Certain non-limiting objectives set forth to meet the aforementioned criteria were:

Objective #1: Preparation of foam formulations using a set of economically viable and environmentally benign surfactants and assess the stability of these formulations in terms of surfactants retaining foaming performances in the presence of disinfectants.

Objective #2: Optimization of the foam characteristics in terms of the formulation-to-water ratio that determines the foam-volume/formulation, foam texture—bubble size, water drainage/evaporation rate form the foam, and the sticking ability of the foam onto slanted and vertical surfaces. Controlling the foam texture that allows uniform deposition of hypochlorite on a surface for complete decontamination and that enables the foam to break/collapse in ˜30 minutes or less.

Objective #3: Assessment of foam deposition using foam sprayer.

A set of example, non-limiting foam formulations were prepared that differed in terms of the surfactant types, and their concentrations. The tasks that were undertaken to meet the objectives, and the inferences made from the results upon execution of the tasks—experiments—are highlighted below.

Objective #1. One set of formulations included a mixture of cocamidopropyl betaine (CAPB) and Sodium dodecyl sulfate (SDS). These surfactants were chosen based on their known foaming properties, and low environmental impact. A particular surfactant, surfactin, was added to these formulations, in view of its prospective antiviral properties. Consideration was given in assessing whether the surface activity property (air/water surface tension) of the surfactants was retained in the presence of hypochlorite; within the concentration range of 0.5 to 10 wt. %.

The surface tension values for these formulations, one of the parametric indicators of the foaming behavior, were assessed regularly (at a 1-week interval) in the presence of hypochlorite. All the formulations were stable in terms of surfactant's activity (surface tension value) being retained for one and a half months period in the presence of hypochlorite.

In the presence of 0.5-1 wt. % CL hypochlorite solution, we noted a moderate increase in the critical micelle concentration for the mixed surfactant systems than that in the absence of hypochlorite. Thus, the foam formulations were stable in terms of surfactants retaining their surface activity for the foam-deployment-on-surface application.

Objective #2. Identifying the foam texture that ensures uniform deposition of hypochlorite on a surface by the foam for deactivation of the virus. The surfactant concentration in the formulations was varied and, air-to-formulation (v/v) ratio, to assess the foam texture. The form texture was described in terms of the average bubble size, and the extent of water drainage. The foam texture was also assessed in terms of foam attachment to the roof and slanted surfaces, and its complete collapse time.

Conclusions for Objective #1 and Objective #2

The prepared formulations exhibited remarkable foamability characteristics, described by the volume of a fine textured (narrow (e.g. 250-800 microns) bubble size distribution) foam formed for a liter of formulation being >20 liters, emphasizing that at least 20 times less amount of hypochlorite could be used in comparison to the conventional approach of spraying a disinfectant formulation in solution form. The texture—bubble size distribution in foam—of foam is a useful indicator of its efficacy in the decontaminating surface, as described earlier. The foam texture was assessed using optical microscopy and found that the foam formed by synthetic surfactants exhibited an average bubble size of 0.7 mm.

The breaking time for some foams was more than one hour, which can be undesirable under certain circumstances. In response, we added PEG (PEG 200, PEG 400, and PEG 800) to the foam formulation at 0.1-0.5 wt. %, which allowed the foam breaking/collapse time to be reduced to ˜30 minutes or less. The foam exhibited remarkable stability—retaining integrity instead of forming patchy regions—upon being deployed onto a surface—glass plate, wooden floor, stone tiles, or walls. The stability was also considered as the period until the foam retained integrity before acquiring a dry form. The foam samples prepared with surfactin, added to the synthetic surfactants in a formulation, resulted in ˜1.2 mm bubble size, larger than the synthetic surfactants alone.

The ultimate result was a foam texture that will minimizes the water drainage and seepage. This foam texture also enabled shorter residence time of water film on a surface after the foam breaks, thus, minimizing fumes generated from hypochlorite solution.

Objective #3. Assessment of Foam Deposition Using Foam Sprayer.

A handheld foam sprayer allowed uniform deposition of foam onto a variety of glass and the tile surfaces, with bubble diameter (˜avg. 1.3 mm) of the foam—foam texture—similar to that obtained through a bench scale foam column. Furthermore, the foam deposited uniformly onto a flat and vertical glass surface (e.g., glass window). The addition of PEG 200 and/or PEG 400 and/or PEG 800 resulted in foam breaking time being reduced desirably from ˜180 minutes to ˜30 minutes.

Additional Disclosure

Introduction

A common mode of the transmission and the spread of viruses such as Covid-19, SARS, West Nile, and Ebola is humans coming in contact with a variety of surfaces. Various studies show that viruses survive on surfaces for a matter of several minutes to days, depending on the physical and chemical compatibility of the surfaces on which they deposit. The deposition of viruses on a surface is considered to be in the form of their composite matrix, e.g., micron-sized saliva droplets containing these viruses. The application of decontaminant solutions for a prolonged period, following guidelines from experts, is necessary to ensure complete deactivation of such a matrix to prevent transmission.

A variety of disinfectants are suggested to be effective by the governmental agencies managing infectious disease control. As per the CDC guidelines, in the USA, the bleach/hypochlorite considered as a potential disinfectant must be deployed on surfaces at 0.5-0.6% chlorine, for at least 2 minutes.

Without being bound to any theory, it was sought to synthesize a bleach/hypochlorite formulation that constituted surfactants, and therefore, enabled deploying of this bleach/hypochlorite onto surfaces in the form of a foam. An advantage of using foam, as opposed to solutions, is much less usage of hypochlorite for decontamination, thus, alleviating the health risk posed to humans—largely caused by inhalation of bleach fumes, and preserving microbiological ecology of the environment by preventing seeping of hypochlorite into the ground.

Success in the application of a hypochlorite foam for decontamination relies on foam delivering sufficient amounts of hypochlorite solution onto the surface to kill the viruses. The delivery can occur through two mechanisms, (1) a continuous hypochlorite solution being in contact with the surface as foam attaches to the surface, and (2) continuous draining of hypochlorite solution from the foam to the surface. The former is important for decontamination of a roof or slanted surface, and the latter for the floors. As per the CDC guidelines, the decontamination fluid should be in contact with the surface for a period spanning somewhere from 2-10 minutes, which could vary depending on the disinfectant types. Thus, it is desirable that the foam largely retains is structural integrity on a surface for an intended duration.

Retention of the integrity, drainage, and collapse are useful key factors for determining the efficacy of foam for decontamination. For example, a fine texture foam can retain its integrity and deposit uniformly, but the disinfectant content could be much lower, with a longer collapse rate for the foam. Alternately, a coarse-grained foam may disintegrate within a few minutes upon deploying onto a surface, but also deposit non-uniformly.

Depending on the foam texture, there is a considerable interplay between drainage and collapse. First, when a foam bubble bursts, the liquid initially present in its walls joins the liquid flowing down in the other walls and the neighboring plateau borders; thus, the draining liquid originates partly in defunct bubbles. As the later part is greater, the foam breaking time is reduced. If the foam is not very stable, both fractions (i.e., drainage and bubble rupture) have comparable magnitudes, and the rate of drainage is complex. Secondly, the probability of bursting generally depends on wall thickness and thus is influenced by drainage. Thirdly, the rate of drainage and the probability of collapse may be influenced by the same property of foam lamellae. Thus, optimization of foam texture adequate for decontamination is largely determined by the water content—air-to-water ratio, consistency, and foam-breaking time, which were assessed.

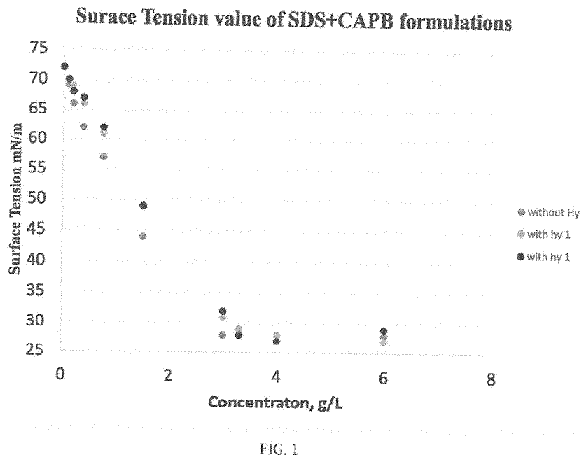

Exemplary surfactants include CAPB, SDS, taurate, and surfactin (FIG. 1). We assessed the stability of these formulations by examining for any changes in the surface tension values and foaming-ability—consistency of foam, foam volume per liter of foam, and foam breaking time. Furthermore, uniformity in hypochlorite deposition in terms of the present of hypochlorite ions content at the interface of the surface and foam was assessed with respect to time during at which foam breakage occurred; this assessment used the Raman spectroscopy technique.

Result and Discussions

Formulations

Two types of formulations were prepared. One set of formulations constituted of synthetically produced surfactants: SDS and CAPB. Another set constituted synthetic and bio surfactants: SDS, CAPB and surfactin. Hypochlorite solution (10-15 wt. % CL) and PEG used in this study were obtained from Sigma Aldrich. Three sprayers were used in these studies: Five Star all-purpose sprayer, Chaplin industrial cleaner degreaser, and Axel 3000.

Surface Activity of Surfactants at Air/Water Interface in the Absence and Presence of Hypochlorite

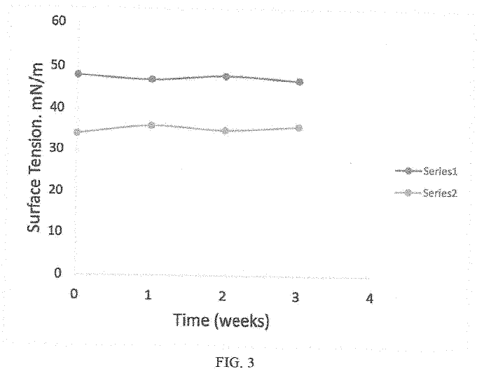

The surface tension values for pure surfactants and their mixtures—formulations—were determined in the presence and absence of hypochlorite (Table 1). The hypochlorite concentration, measured as chlorine content, differed from 0.1-10%. The surface tension values for CAPB, taurate, SDS, and surfactin determined after 24 hours of preparation of these solutions, and in the absence and presence of hypochlorite are given in Table 1 and FIGS. 1, 2, and 3; Taurate was used for fundamental reasons and was not considered in preparing the final formulation(s). (The surface tension values were determined using a tensiometer.)

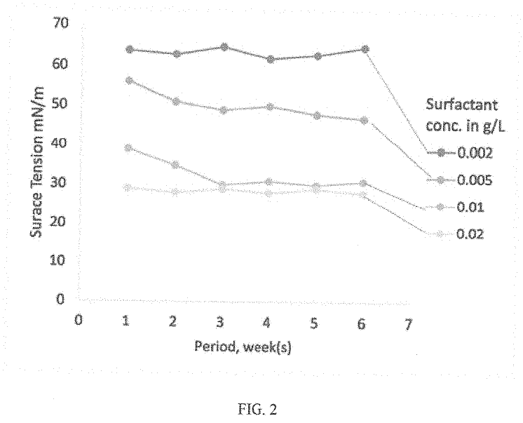

The CMC values for these surfactants in the absence of hypochlorite, were as reported in the literature. In the presence of hypochlorite, the CMC values for SDS and Taurate increased slightly (FIG. 2), whereas, for CAPB, a small decrease or no change in the CMC value was be noted. These studies indicated that the surface-active properties of the surfactants were not affected as anticipated to occur due to surfactant degradation by hypochlorite, even at high CL (˜10 wt. %) concentrations. The hypochlorite concentration, however, was found to decrease and in the formulation, 30-40%, after 24 hrs. A chlorine strip was used to determine hypochlorite degradation.

Foamability Study

While applying bleach solution for surface decontamination, the amount of water-retaining on a surface varies depends on, e.g., the surface tension dependent minimum thickness of water on a surface, the flatness of the surface, and the presence of pores and cracks. Whereas, upon depositing a foam onto a surface, the thickness of the film developing on the surface depends on the water-to-air ratio of the foam.

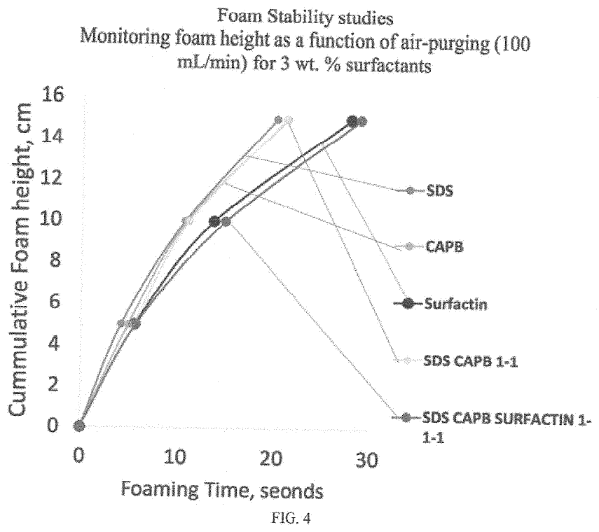

We first assessed the formation of a uniformly textured foam by varying the air-to-water ratio, achieved through controlling the water flow rate 50-100 mL/min; the surfactant concentration was in the range of 1-3 wt. %. FIG. 4 shows the foamability rate for the cases where a consistent foam was formed.

The foamability rate, defined as foam height per min, was found to increase for surfactants and their mixtures as CAPB>SDS>CAPB+SDS>CAPB+SDS+surfactin >surfactin. The foam-breaking rates were assessed in terms of foam breaking time upon placing the foam on a glass slide. The foam breaking rate was found to as CAPB>SDS>CAPB+SDS>CAPB+SDS+surfactin >surfactin. Thus, this study provided a parametric assessment of foamability and foam stability.

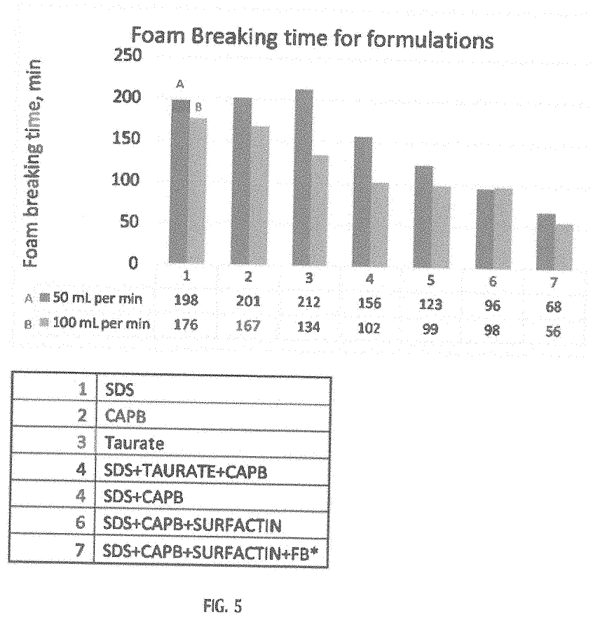

The foam breaking time was a parameter of interest, as it determines the hypochlorite and surfactant's contact time with the surface. While it can be desirable that the foam is stable for more than 10 minutes to ensure viruses and bacteria being submerged in a thin film formed upon deposition of foam, it is also desirable that the foam breaks/disappears after its intended virus killing action is completed. The foam breaking time was determined for the foams prepared with different formulations, and that after depositing on the glass side, tiles, wood, and plastic. One may note that for SDS+CAPB and SDS+CAPB+surfactin formulations, the foam braking time—complete dry foam was ˜180 and 120 minutes (FIG. 5). This breaking time was considered large for the application of the foam for virus decontamination. Therefore, to reduce the foam breaking time, PEG was used; PEG 200, PEG 400 and PEG 8000 in different ratios (typically 10:8:0.5 by weight). We found that the breaking time decreased upon adding PEG to the formulations, with breaking time being reduced to −30 mins with PEG concentration being ˜0.25 wt. %.

Bubble Size Distribution of the Foams

The foam formed by the surfactant formulations was assessed in terms of the bubble size distribution, from the time the foam was formed until it completely dried out—very dry foam. These foam samples were prepared following the optimized—consistent foam (smallest to largest bubble size differing within 2 mms and more than 50% bubbles with average bubble size)—data obtained for air-flow rate (100 mL/min) and surfactant concentration (3 wt. %) from foam stability studied.

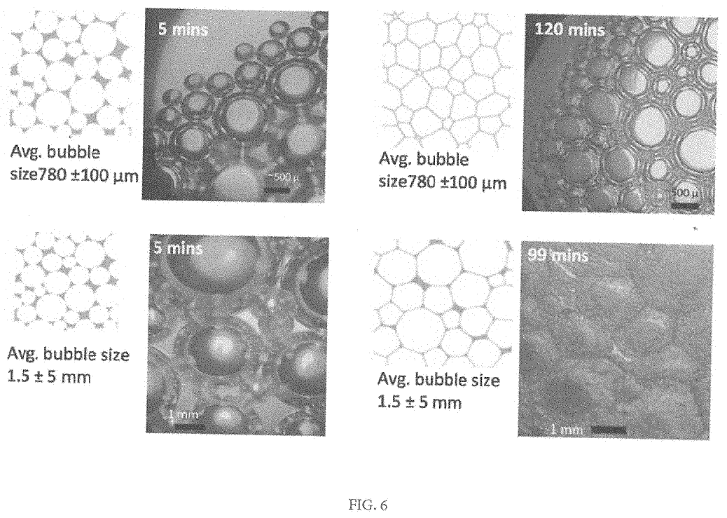

FIG. 6 showing bubble size distribution data for SDS+CAPB and SDS+CAPB+surfactin systems indicated that surfactin addition to the foam formulation resulted in almost 3-4 times increase in the bubble size. While finer sized bubbles—avg. 500 micron—bubbles were formed with the SDS+CAPB formulation, the bubbles were on an avg. 2 mm for SDS+CAPB+surfactin systems. The addition of surfactin resulted in residues being left on the glass slides.

Foam Deposition Using Sprayer

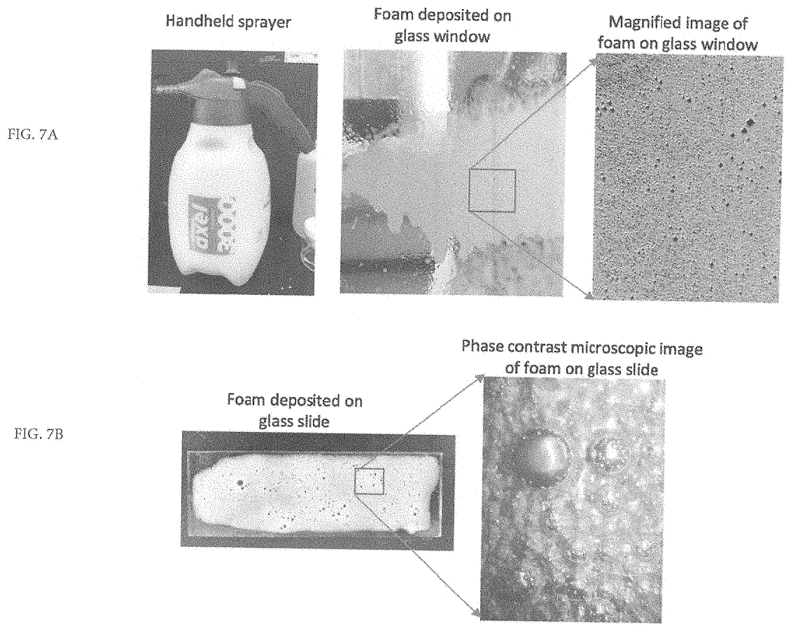

A household sprayer was used to deposit foam onto the glass and tile (floor) surfaces. Three nozzle sizes—generating bubbles at different average diameters—were used for foam generation (FIG. 7). It was found that spraying foam onto the glass surface resulted in the deposition of the foam in the form of a uniform layer. The foams deposited onto the glass slides and floor (tiles) were assessed for bubble sizes and bubble breaking time. The average bubble sizes—1.7 mm—were found to be 20% smaller than that generated using the foam column. However, the foam breaking time—˜35 mins—for the formulations were found to be within 10% differences from that formed with foam column.

Spectroscopic Assessment of Bleach Deposition on Surface, and Degradation

To monitor degradation of hypochlorite in the formulations (liquids), foams, and deposit residues, one can use Raman microscopy. Raman spectra were acquired using a Horiba Aramis confocal Raman microscope with a 10× objective (numerical aperture 0.25), a 1200 grooves/mm diffraction grating, and a 17-mW 633-nm HeNe laser without attenuation. The monochromator was calibrated before each set of measurements using the Si peak at 520.7 cm-1 of a Si wafer reference sample.

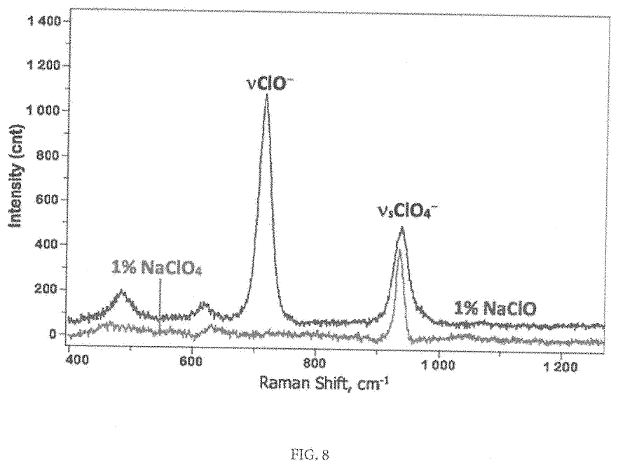

The degradation of hypochlorite ion (ClO—) was assessed by the intensity ratio of the main Raman peaks of ClO— at 715 cm-1 and perchlorate ClO4- at 937 cm-1 (FIG. 8). Perchlorate is a product of the final degradation of hypochlorite. The ClO—/ClO₄— peak ratio was about 2, indicating that about 30% of hypochlorite from Aldrich is already degraded in a freshly prepared 1% solution in water.

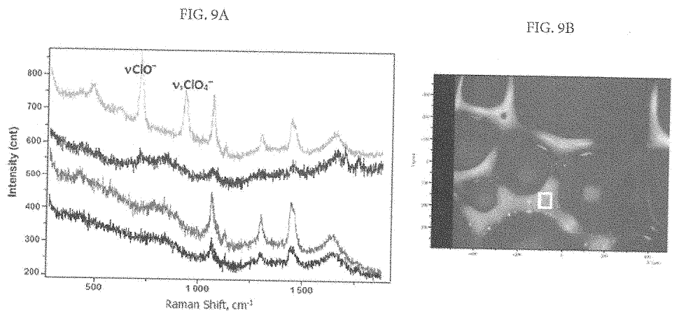

Using the ClO—/ClO₄— peak ratio, we found that activity of hypochlorite ion in the top of a foam freshly prepared from a formulation that contained 1.5 wt % SDS+1.5% wt CAPB+1% Aldrich hypochlorite (assuming that it contains 10% hypochlorite)) decreased in 30 min from 2 to 1 (FIG. 9B). This ratio is 1.5 in the dry residue left on the slide in 90 min after the deposition of the foam on a glass slide. This ratio is significantly higher than in the top of the foam, suggesting that degradation of hypochlorite inside the foam is slower than within its top surface. For comparison, FIG. 9A also shows Raman spectra of a foam and residue of the same surfactant mixture but without hypochlorite (1.5 wt % SDS+1.5% wt CAPB).

These spectra do not exhibit peaks in the spectral region of the analytical peaks of ClO— and ClO4-. A similar result has been obtained for formulation (1.5 wt SDS+1.5 wt LAPB+0.25 wt % PEG+1 wt % NaClO). We also found that the distribution of hypochlorite within the dry residue is homogeneous.

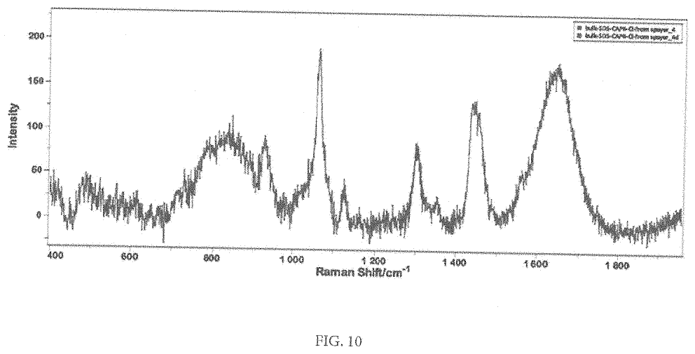

At the same time, it was found that activity of hypochlorite significantly degrades after storage of the formulations for 24 h. FIG. 10 shows that a Raman spectrum of the 24-h aged formulation (1.5 wt. % SDS+1.5 wt. % LAPB+1 wt. % NaClO) has only traces of the ClO— peak. This result suggests that the most effective approach to disinfect using the developed formulation is to mix the bleach and surfactants within an hour before the application.

### SUMMARY

Without being bound to any particular theory or embodiment, some non-limiting conclusions are given below.

1. A set of stable foam formulations were prepared using SDS and CAPB as the main surfactants.

2. A novel material, hypochlorite gel/cream, was synthesized, which constituted SDS and CAPB as the main surfactants at a total concentration of 35 wt. %.

3. An exemplary foam texture was developed that minimizes the water drainage followed by seepage, enabling a shorter residence time of water film on a surface after the foam breaks, thus, minimizing fumes generated from the hypochlorite solution. We also gained an understanding of the foam texture—known as the transition regime of semi-wet-to-dry foam texture—that is ideal for the application of foam formulation.

4. The foam prepared here deposits onto flat and slanted surfaces (glass, stone tiles, and wooden floors) without spontaneous drainage of water, and with almost zero flowability. Microscopy examination of this foam suggested the foam texture exhibiting the characteristic of wet foam. However, water drainage and flowability—laterally or vertically, or dripping from a roof—was negligible because of the high viscosity (5+1 mPa·s) of the liquid phase in the lamellar region of the foam. Without being bound to any particular theory, this viscosity may be due to the formation of worm-like micelles for the surfactant in the lamellar region and also due to PEGs, present as a viscosity modifier.

Table 1: Surface tension values of surfactants and their mixture in the absence and presence of hypochlorite (0.7-1 wt. % CL)

| | | No | Min | 0.7-1% | Min |
|---|---|---|---|---|---|
| | | hypo- | surface | hypo- | surface |
| | | chlorite | tension | chlorite | tension |
| SDS | | 2.4 g/L | 28 mN/m | 2.6 g/L | 28 mN/m |
| CAPB | | 0.3 g/L | 28 mN/m | 0.31 g/L | 28 mN/m |
| SDS + | 1:1 | .17 g/L | 28 mN/m | 0.19 g/L | 28 mN/m |
| CAPB | | | | | |
| SDS + | 1:1:1 | 2.1 g/L | 29 mN/m | 2.3 g/L | 29 mN/m |
| CAPB + | | | | | |
| SURFACTIN | | | | | |

Aspects

The following Aspects are illustrative only and do not necessarily limit the scope of the present disclosure or the appended claims.

Aspect 1. An aqueous foam, comprising: at least one surfactant; at least one defoamer; and optionally, at least one disinfectant, the at least one surfactant, the at least one defoamer, and the at least one optional disinfectant being present such that (a) the foam is characterized as breaking within about 30 minutes of application to a surface under exposure to ambient conditions (e.g., 101.325 kPa, 20 deg C. and 50% humidity), (b) the foam exhibits an essentially uniform deposition along a surface to which the foam is applied, (c) the foam forms a fluid layer at an interface between the foam and a surface to which the foam is applied, (d) any combination of (a), (b), and (c).

The foam can have surfactant present at from, e.g., about 1 to about 50 wt %, from about 2 to about 49 wt %, from about 3 to about 47 wt %, from about 4 to about 46 wt %, from about 5 to about 45 wt %, from about 6 to about 45 wt %, from about 7 to about 44 wt %, from about 8 to about 43 wt %, from about 9 to about 42 wt %, from about 10 to about 41 wt %, from about 1 to about 40 wt %, from about 11 to about 39 wt %, from about 12 to about 38 wt %, from about 13 to about 37 wt %, from about 14 to about 36 wt %, from about 15 to about 35 wt %, from about 16 to about 34 wt %, from about 16 to about 34 wt %, from about 17 to about 35 wt %, from about 17 to about 33 wt %, from about 18 to about 32 wt %, from about 19 to about 31 wt %, from about 20 to about 30 wt %, from about 21 to about 29 wt %, from about 22 to about 28 wt %, from about 23 to about 27 wt %, from about 24 to about 26 wt %, or even about 25 wt %. Surfactant levels in the range of from about 1 to about 10 wt % (and all intermediate values and sub-ranges) are considered suitable, but are not required. A foam can include a single surfactant, but can also include a plurality of surfactants.

The defoamer can be present at, e.g., from about 0.05 to about 1 wt %, including all intermediate values and sub-ranges, e.g., from about 0.05 to about 1 wt %, from about 0.1 to about 0.95 wt %, from about 0.15 to about 0.90 wt %, from about 0.2 to about 0.85 wt %, from about 0.25 to about 0.80 wt %, from about 0.3 to about 0.75 wt %, from about 0.35 to about 0.70 wt %, from about 0.4 to about 0.65 wt %, from about 0.45 to about 0.60 wt %, from about 0.50 to about 0.55 wt %, and all intermediate values and sub-ranges. The defoamer can be, e.g., PEG (such as, for example, PEG 200, PEG 200 DO, PEG 400, PEG 400 DO, PEG 800, PEG 800 DO), insoluble oils, polydimethylsiloxanes and other silicones, alcohols, stearates, glycols, and the like.

Disinfectant can be present at, e.g., from about 0.1 to about 20 wt %. For example, the disinfectant can be present at, e.g., from about 0.1 to about 20 wt %, from about 0.2 to about 19 wt %, from about 0.3 to about 18 wt %, from about 0.4 to about 17 wt %, from about 0.5 to about 16 wt %, from about 0.6 to about 15 wt %, from about 0.7 to about 14 wt %, from about 0.8 to about 13 wt %, from about 0.9 wt %, or even from about 1 to about 12 wt %. Disinfectant levels of from about 1 to about 10 wt % are considered especially suitable. In the case where sodium hypochlorite is present as a disinfectant, the level of sodium disinfectant can be measured in terms of chlorine content.

Aspect 2. The foam of Aspect 1, wherein the disinfectant comprises at least one of sodium hypochlorite, ammonia, a benzalkonium chloride, or any combination thereof.

A benzalkonium chloride can have, e.g., the following formula:

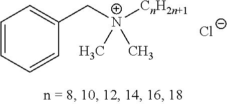

Aspect 3. The foam of Aspect 2, wherein the disinfectant comprises sodium hypochlorite.

Aspect 4. The foam of any one of Aspects 1 to 3, wherein the at least one surfactant comprises sodium dodecyl sulfate (SDS), one or more taurates, cocamidopropyl betaine (CAPB), surfactin, sodium caproyl isethionate (SCI), sodium capryloyl isethionate (SCI), sodium lauroyl isethionate (SLI), sodium palmitoyl isethionate (SPI). sodium laureth sulfate (SLES), sodium lauryl sulfate (SLS) or any combination thereof.

Aspect 5. The foam of Aspect 4, wherein the at least one surfactant comprises SDS.

Aspect 6. The foam of any one of Aspects 1 to 5, wherein the at least one defoamer comprises polyethylene glycol (PEG). PEG can have a molecular weight in the range of from, e.g., about 200 to about 8000.

Aspect 7. The foam of any one of Aspects 1 to 6, wherein the at least one disinfectant is present at from about 0.2 wt % to about 2 wt %. In some embodiments, the at least one disinfectant is present at from about 0.5 to about 1.5 wt %, which range is considered especially suitable when the disinfectant is sodium hypochlorite.

Aspect 8. The foam of any one of Aspects 1 to 7, wherein the foam exhibits an average bubble size of from about 250 μm to about 4 mm, e.g., from about 250 μm to about 4 mm, from about 300 μm to about 3.5 mm, from about 350 μm to about 3.0 mm, from about 400 μm to about 2.5 mm, from about 500 μm to about 2 mm, or even about 1 mm.

Aspect 9. The foam of Aspect 8, wherein the foam exhibits an average bubble size of from about 500 μm to about 3 mm.

Aspect 10. The foam of any one of Aspects 1 to 9, further comprising a viscosity modifier. Example such viscosity modifiers include, e.g., water-soluble polymer PAMs (polyacrylamides) and poly(acrylic acid) (PAA; e.g., Carbomer™). A foam according to the present disclosure can be, e.g., shear-thinning or shear-thickening.

Aspect 11. The foam of any one of Aspects 1 to 10, wherein the foam effects an essentially uniform application of the disinfectant to a surface to which the foam is applied.

Aspect 12. The foam of any one of Aspects 1 to 11, wherein during breaking, the foam remains in an essentially unchanged position. As an example, the foam can be characterized as not slipping along a vertical surface to which the foam is applied. It should also be understood that a foam according to the present disclosure can effect little to no aerosol formation when the foam is applied from a sprayer or nozzle. Without being bound to any particular theory, the presence of a viscosity modifier can assist in reducing aerosol formation.

Aspect 13. A method, comprising: formation of a foam according to any one of Aspects 1 to 12, wherein the formation of the foam comprises foaming a foamable base formulation; and effecting deposition of the foam onto a surface, the deposition of the foam optionally effecting disinfection of the surface.

The deposition can be effected by, e.g., spraying, pouring, swabbing. Spraying is considered especially suitable.

Aspect 14. The method of Aspect 13, wherein a line extending perpendicular from the surface is inclined at from 0 to 180 degrees relative to gravity, e.g., at from 0 to about 180 degrees, from 0 to about 170 degrees, from 0 to about 160 degrees, from 0 to about 150 degrees, from 0 to about 140 degrees, from 0 to about 130 degrees, from 0 to about 130 degrees, from 0 to about 120 degrees, from 0 to about 110 degrees, from 0 to about 100 degrees, from 0 to about 90 degrees, from 0 to about 80 degrees, from 0 to about 70 degrees, from 0 to about 60 degrees from 0 to about 50 degrees, from 0 to about 40 degrees, from 0 to about 30 degrees, from 0 to about 20 degrees, from 0 to about 10 degrees. For example, the surface can be vertical, e.g., a wall or window. A surface can also be inclined, e.g., a sloping wall. A surface can also be a ceiling or a floor.

Aspect 15. The method of Aspect 13, wherein the surface is characterized as a wall, a ceiling, a window, a floor, a wall covering, a floor covering, or any combination thereof.

Aspect 16. The method of Aspect 13, wherein the surface is a pervious medium, and wherein deposition solubilizes an oil disposed on or in the pervious medium.

Aspect 17. The method of Aspect 16, wherein the pervious medium is a soil or carpet.

Aspect 18. A foamable base formulation, comprising: at least one surfactant; at least one defoamer; and optionally, at least one disinfectant, the at least one optional disinfectant, the at least one surfactant, and the at least one defoamer being present such that the foamable formulation is capable of being foamed so as to give rise to a foam, e.g., a foam according to any one of Aspects 1 to 12. The at least one surfactant can be present at, e.g., from about 1 wt % to about 50 wt %, and all intermediate values.

Aspect 19. The foamable formulation of Aspect 18, wherein (a) the foamable formulation exhibits an essentially unchanged surface tension over a period of about 2 weeks when stored at ambient conditions, (b) essentially no aerosol forms when the foamable base formulation is foamed, or both (a) and (b). The formulation can exhibit an essentially unchanged surface tension for, e.g., 1, 2, 3, 4, 5, 6, or even 7 weeks. Without being bound to any particular theory or embodiment, the presence of a viscosity modifier can assist in reducing or even eliminating the formation of aerosol when the foamable formulation is foamed.

Aspect 20. A system, comprising: a vessel (e.g., a tank) configured to contain a foamable base formulation according to any one of Aspects 18 to 19; and an aeration train, the aeration train configured to effect aeration of the foamable base formulation so as to give rise to an aqueous foam according to any one of Aspects 1 to 12. The system can include a pump (automated or manual) configured to aerate the foamable base formulation. A system can include a source of propellant; the propellant can be air.

What is claimed:

1. An aqueous foam, comprising:
at least one surfactant;
at least one defoamer; and
optionally, at least one disinfectant,
the at least one surfactant, the at least one defoamer, and the at least one optional disinfectant being present such that
(a) the foam is characterized as breaking in less than about 30 minutes of application to a surface under exposure to ambient conditions,
(b) the foam exhibits an essentially uniform deposition along a surface to which the foam is applied,
(c) the foam forms a fluid layer at an interface between the foam and a surface to which the foam is applied,
(d) any combination of (a), (b), and (c).

2. The foam of claim 1, wherein the disinfectant comprises at least one of sodium hypochlorite, ammonia, benzalkonium chloride, or any combination thereof.

3. The foam of claim 2, wherein the disinfectant comprises sodium hypochlorite.

4. The foam of claim 1, wherein the at least one surfactant comprises sodium dodecyl sulfate (SDS), taurates, cocamidopropyl betaine (CAPB), surfactin, sodium caproyl isethionate (SCI), sodium capryloyl isethionate (SCI), sodium lauroyl isethionate (SLI), sodium palmitoyl isethionate (SPI). sodium laureth sulfate (SLES), or any combination thereof.

5. The foam of claim 4, wherein the at least one surfactant comprises SDS.

6. The foam of any claim 1, wherein the at least one defoamer comprises polyethylene glycol (PEG).

7. The foam of claim 1, wherein the at least one disinfectant is present at from about 0.2 wt % to about 2 wt %

8. The foam of claim 1, wherein the foam exhibits an average bubble size of from about 250 μm to about 4 mm.

9. The foam of claim 8, wherein the foam exhibits an average bubble size of from about 500 μm to about 3 mm.

10. The foam of claim 1, further comprising a viscosity modifier.

11. The foam of claim 1, wherein the foam effects an essentially uniform application of the disinfectant to a surface to which the foam is applied.

12. The foam of claim 1, wherein during breaking, the foam remains in an essentially unchanged position on a surface to which the foam is applied.

13. A method, comprising:
formation of a foam according to claim 1, wherein the formation of the foam comprises foaming a foamable base formulation; and
effecting deposition of the foam onto a surface, the deposition of the foam optionally effecting disinfection of the surface.

14. The method of claim 13, wherein a line extending perpendicular from the surface is inclined at from 0 to 180 degrees relative to gravity.

15. The method of claim 13, wherein the surface is characterized as a wall, a ceiling, a window, a floor, a wall covering, a floor covering, or any combination thereof.

16. The method of claim 13, wherein the surface is a pervious medium, and wherein deposition solubilizes an oil disposed on or in the pervious medium.

17. The method of claim 16, wherein the pervious medium is a soil or carpet.

18. A foamable base formulation, comprising:
at least one surfactant;
at least one defoamer; and
optionally, at least one disinfectant,
the at least one optional disinfectant, the at least one surfactant, and the at least one defoamer being present such that the foamable formulation is capable of being foamed so as to give rise to a foam according to claim 1.

19. The foamable formulation of claim 18, wherein (a) the foamable formulation exhibits an essentially unchanged surface tension over a period of about 2 weeks when stored at ambient conditions, (b) essentially no aerosol forms when the foamable base formulation is foamed, or both (a) and (b).

20. A system, comprising:
a vessel configured to contain a foamable base formulation according to claim 18; and
an aeration train, the aeration train configured to effect aeration of the foamable base formulation so as to give rise to an aqueous foam according to claim 1.
